(19) United States
(12) Patent Application Publication
(10) Pub. No.: US 2021/0093671 A1
(43) Pub. Date: Apr. 1, 2021

(54) USE OF MICROVESICLES DERIVED FROM STEM CELLS IN TREATMENT OF NEUROINFLAMMATION, PARTICULARLY INDUCED BY STROKE

(71) Applicant: INSTYTUT MEDYCYNY DOSWIADCZALNEJ I KLINCZNEJ IM.M. MOSSAKOWSKIEGO PAN, Warszawa (PL)

(72) Inventors: Anna ANDRZEJEWSKA, Grodzisk Mazowiecki (PL); Sylwia DABROWSKA, Warszawa (PL); Miroslaw JANOWSKI, Grabow nad Pilica (PL); Barbara LUKOMSKA, Warszawa (PL)

(21) Appl. No.: 17/051,892

(22) PCT Filed: Apr. 30, 2019
(86) PCT No.: PCT/PL2019/050027

§ 371 Date: Oct. 30, 2020

(30) Foreign Application Priority Data
Apr. 30, 2018 (PL) P.425406

(51) Int. Cl. A61K 35/28; A61P 29/00; A61P 25/00

(52) U.S. Cl. CPC A61K 35/28; A61P 25/00; A61P 29/00

(57) ABSTRACT

Use of microvesicles derived from stem cells in treatment of encephalitis, in particular induced by stroke, is disclosed.

The present invention relates to a method of treatment of neuroinflammation and to a medicament applicable in treatment of neuroinflammation in mammals, in particular in humans, especially in patients after stroke.

Brain stroke causes a variety of dysfunctions of nervous tissue, in particular stroke-induced neuroinflammation. Nervous tissue ischemia induced by stroke leads to a decrease in the amount of oxygen and glucose reaching brain cells. The resulting reduction in the amount of available energy causes an increase in glutamate release, activation of receptors thereof, which in consequence leads to cell membrane depolarization in the vicinity of the ischemia. The resulting change in Na⁺, K⁺, Ca²⁺ and Cl⁻ ion concentrations in extra- and intracellular space leads to brain edema and development of encephalitis and at further stages to eliciting molecular mechanisms resulting in cell death. This leads to irreversible damage of brain tissue affected by the stroke.

Therefore, it is very desirable to provide means that could be useful for inhibiting and/or limiting stroke-induced neuroinflammation.

Previous attempts of utilizing extracellular vesicles (EVs) for this purpose, released from mesenchymal stem cells, in particular human bone marrow derived mesenchymal stem cells (hBM-MSCs), have not brought desired results. In particular, intravenous administration of EVs from hBM-MSCs has not elicited neuroimmunomodulatory activity against stroke-induced neuroinflammation (THORSTEN R. DOEPPNER et al. STEM CELLS TRANSLATIONAL MEDICINE 2015; 4:1131-1143, see page 1135, last paragraph of the summary of results).

In the research that lead to obtaining the present invention it was surprisingly found that intra-arterial administration of microvesicles derived from stem cells reduces stroke-induced neuroinflammation.

In particular, it was found that such use of EVs from hBM-MSCs reduces the number of inflammatory cells such as: activated microglia (ED1) and leucocytes (CD45) and proinflammatory molecules such as: IL-1alpha, IL-1beta, IL-6, IL-4, IFN-Gamma, CXCL1, MIP-1alpha, MIP-3alpha and MCP-1.

The object of the invention are microvesicles derived from mesenchymal stem cells for use as an immunomodulating medicament administered intra-arterially in treatment of neuroinflammation, in particular induced by stroke.

Preferably, the medicament obtained according to the invention, comprising microvesicles derived from mesenchymal stem cells, is administered intra-arterially while performing a procedure of intra-arterial clot removal.

Preferably, microvesicles for use according to the invention are used for inducing one of the following immunomodulating effects: reduction of the number of inflammatory cells such as: activated microglia (ED1) and leukocytes (CD45) or proinflammatory molecules such as: IL-1alpha, IL-1beta, IL-6, IL-4, IFN-Gamma, IL-10, CXCL1, MIP-1alpha, MIP-3alpha and MCP-1.

Preferably, microvesicles for use according to the invention are extracellular vesicles (EVs) released from human mesenchymal stem cells, in particular from bone marrow (hBM-MSCs).

Another object of the invention is a pharmaceutical composition for treatment of neuroinflammation, in particular induced by stroke, characterized by the fact it comprises microvesicles derived from stem cells, wherein the composition is for intra-arterial administration.

Preferably, the pharmaceutical composition according to the invention comprises microvesicles for therapeutic use as defined above.

According to the invention, microvesicles administered intra-arterially are used for treatment of patients with brain stroke acting via reduction of neuroinflammation.

In view of the fact that currently the approach to treatment of stroke affected brain is changing and intra-arterial clot removal is performed more and more willingly, which improves the treatment results reducing the stroke that nevertheless usually occurs, thus administering microvesicles during the same procedure or administering these afterwards or administration in patients that do not qualify for clot removal, should provide a further desired therapeutic effect.

Changes in the approach to stroke treatment have simultaneously evidenced that intra-arterial procedures are safe in this type of patients. This is a further incentive for intra-arterial administration of microvesicles also in patients affected by stroke for whom intra-arterial clot removal is not an option (approximately half of patients). Intra-arterial administration also has other advantages in comparison with other administration routes, as it allows reaching directly and precisely the stroke-affected brain area. This provides for limiting the effective dose inducing a beneficial immunomodulating effect in the tissue affected by inflammation in comparison to systemic administration.

In order to better explain the concept thereof, the invention has been demonstrated in the examples hereinbelow, additionally illustrated on the following figures:

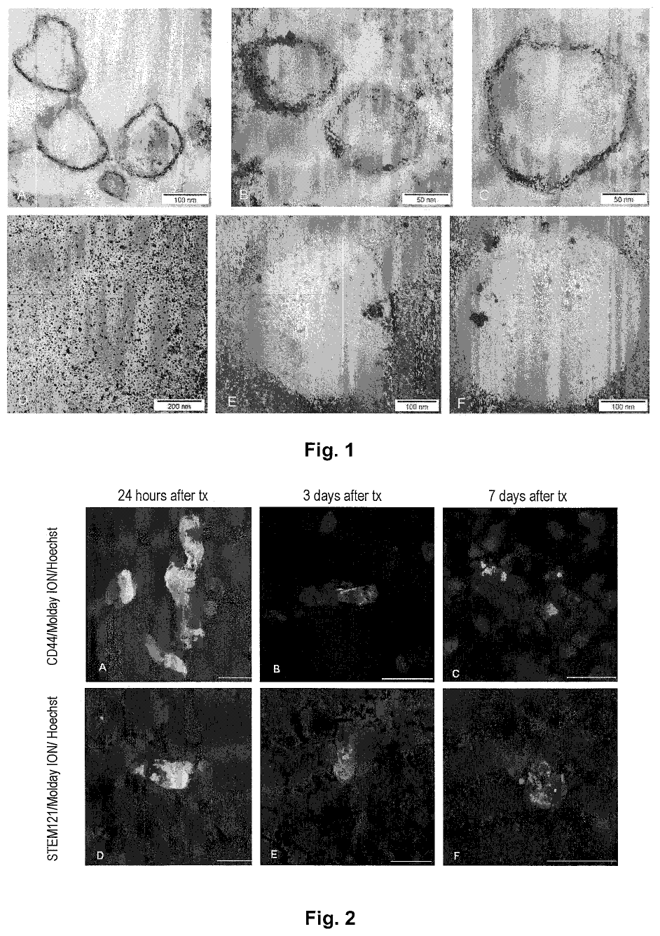

FIG. 1 shows extracellular vesicles after their isolation from native hBM-MSCs (A-C), iron nanoparticles conjugated with' rhodamine B (D) and EVs isolated from cells labeled with Molday ION (E-F). A representative view as observed under a transmission electron microscope shows a heterogenous EVs population consisting of larger and smaller microvesicles (A-C), iron nanoparticles are present inside EVs after their isolation from Molday ION-labelled hBM-MSCs (E-F).

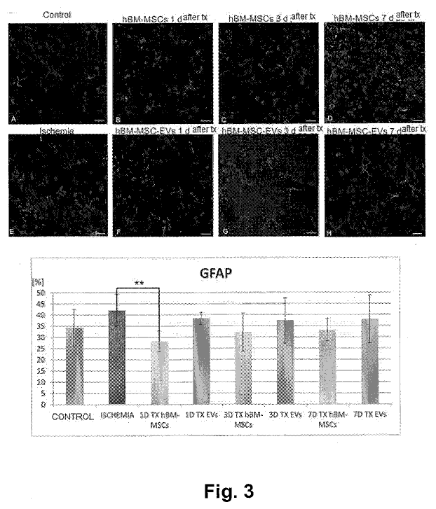

FIG. 2 shows a view under a confocal microscope of hBM-MSCs labelled in vivo with Molday ION (red color), visible in the right hemisphere of rat brain after intra-arterial transplantation. Immunohistochemical analysis of the cells after using antibodies: anti-CD44, specific for MSC cells (A-C) and anti-STEM121, a human cell marker (D-F) (green color). Cell nuclei were additionally labelled with Hoechst dye 33258 (blue color). Scale of 20 μm.

FIG. 3 shows an immunohistochemical analysis of astrocytes in the right brain hemisphere of: a healthy rat (A), 48 hours after striatum was damaged with ouabain (E) and after 1, 3 and 7 days from intra-arterial transplantation of hBM-MSCc (B-D) or EVs (F-H) thereafter. The graph shows a percentage of GFAP⁺ cells in individual animal groups. Results are shown as mean values±SD, n=6, ** p<0.01.

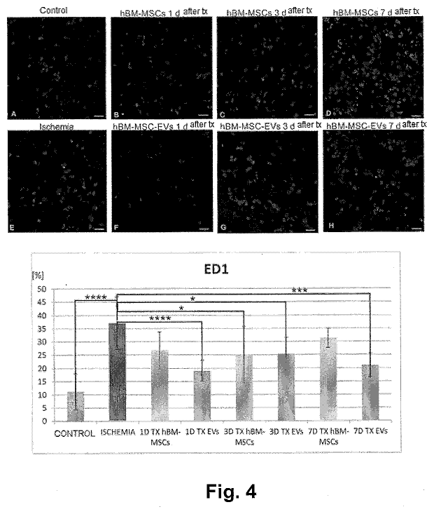

FIG. 4 demonstrates an immunohistochemical analysis of microglial cells in the right brain hemisphere of: a healthy rat (A), 48 hours after striatum was damaged with ouabain (E) and after 1, 3 and 7 days from intra-arterial transplantation of hBM-MSCc (B-D) or EVs (F-H) thereafter. The graph shows a percentage of ED⁺ cells in individual animal groups. Results are shown as mean values±SD, n=6, *p<0.05; ***p<0.01; ****p<0.0001.

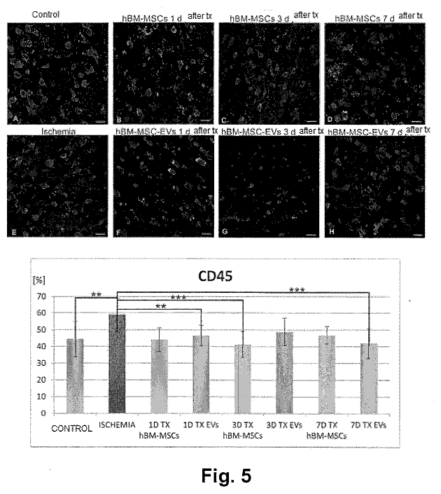

FIG. 5 demonstrates an immunohistochemical analysis of leucocytes in the right brain hemisphere of: a healthy rat (A), 48 hours after striatum was damaged with ouabain (E) and after 1, 3 and 7 days from intra-arterial transplantation of hBM-MSCc (B-D) or EVs (F-H) thereafter. The graph shows a percentage of CD45RA⁺ cells in individual animal groups. Results are shown as mean values±SD, n=6, *p<0.05; **p<0.01; ***p<0.001.

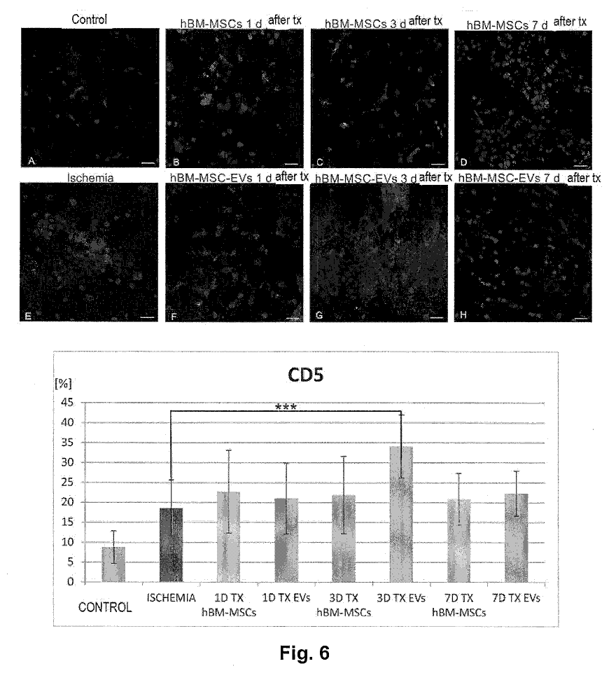

FIG. 6 demonstrates an immunohistochemical analysis of T lymphocytes in the right brain hemisphere of: a healthy rat (A), 48 hours after striatum was damaged with ouabain (E) and after 1, 3 and 7 days from intra-arterial transplantation of hBM-MSCc (B-D) or EVs (F-H) thereafter. The graph shows a percentage of CD5⁺ cells in individual animal groups. Results are shown as mean values±SD, n=6, ***p<0.001.

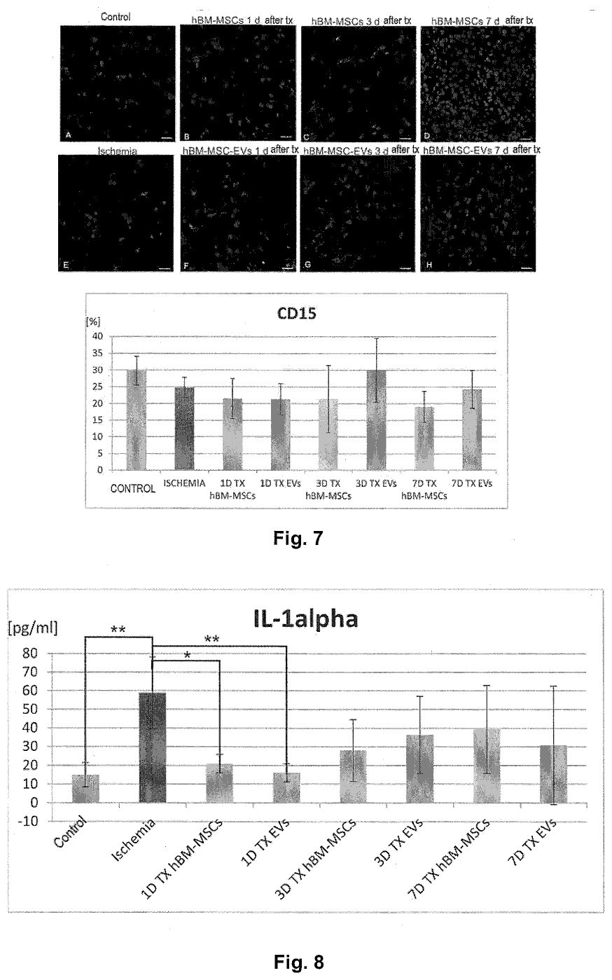

FIG. 7 demonstrates an immunohistochemical analysis of neutrophils in the right brain hemisphere of: a healthy rat (A), 48 hours after striatum was damaged with ouabain (E) and after 1, 3 and 7 days from intra-arterial transplantation of hBM-MSCc (B-D) or EVs (F-H) thereafter. The graph shows a percentage of CD15⁺ cells in individual animal groups. Results are shown as mean values±SD, n=6.

FIG. 8 shows interleukin 1α levels in the right brain hemisphere in individual experimental rat groups. Results are shown as mean values±SD, n=6, *p<0.05; **p<0.01; ***p<0.001.

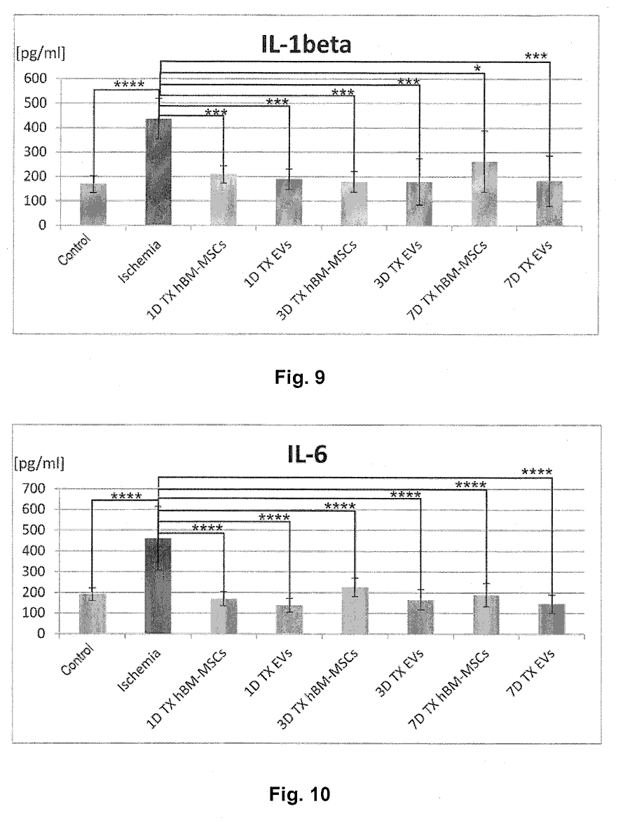

FIG. 9 shows interleukin 1β levels in the right brain hemisphere in individual experimental rat groups. Results are shown as mean values±SD, n=6, *p<0.05; ***p<0.001; ****p<0.0001.

FIG. 10 shows interleukin 6 levels in the right brain hemisphere in individual experimental rat groups. Results are shown as mean values±SD, n=6, ****p<0.0001.

FIG. 11 shows interleukin 4 levels in the right brain hemisphere in individual experimental rat groups. Results are shown as mean values±SD, n=6, *p<0.05; **p<0.01.

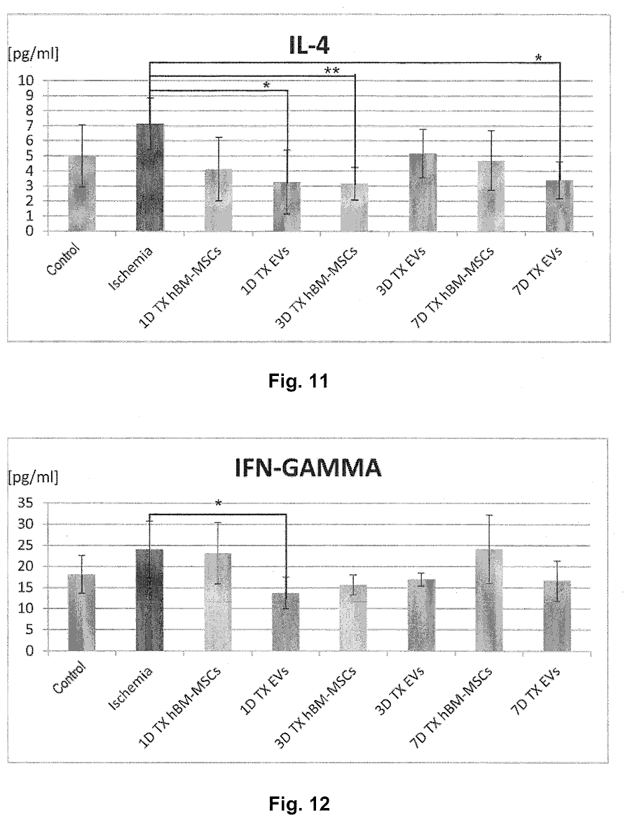

FIG. 12 shows interferon gamma levels in the right brain hemisphere in individual experimental rat groups. Results are shown as mean values±SD, n=6, *p<0.05.

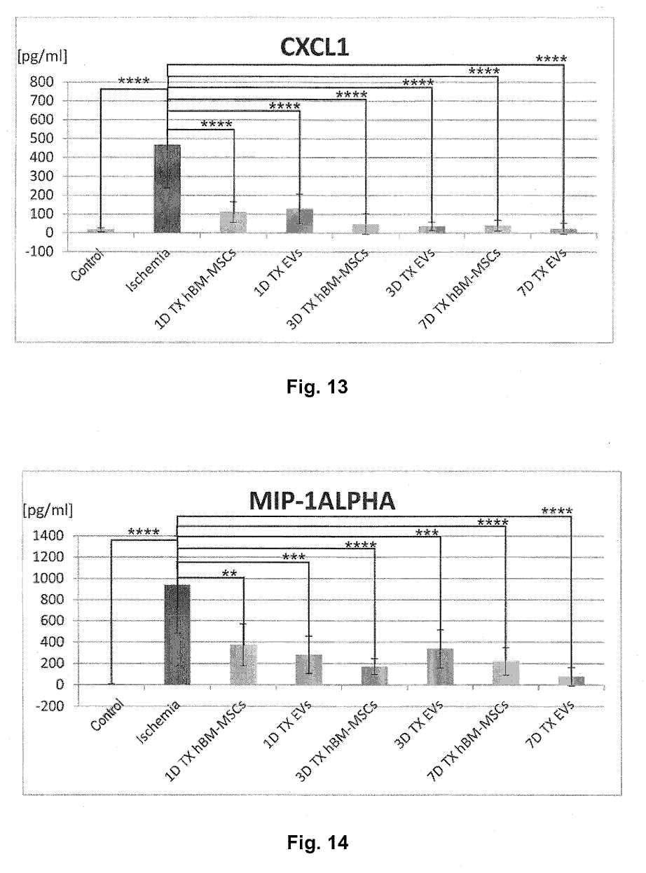

FIG. 13 shows the levels of CXC chemokine ligand 1 in the right brain hemisphere in individual experimental rat groups. Results are shown as mean values±SD, n=6, ****p<0.0001.

FIG. 14 shows the levels of macrophage inflammatory protein 1 alpha in the right brain hemisphere in individual experimental rat groups. Results are shown as mean values±SD, n=6, **p<0.01; ***p<0.001; ****p<0.0001.

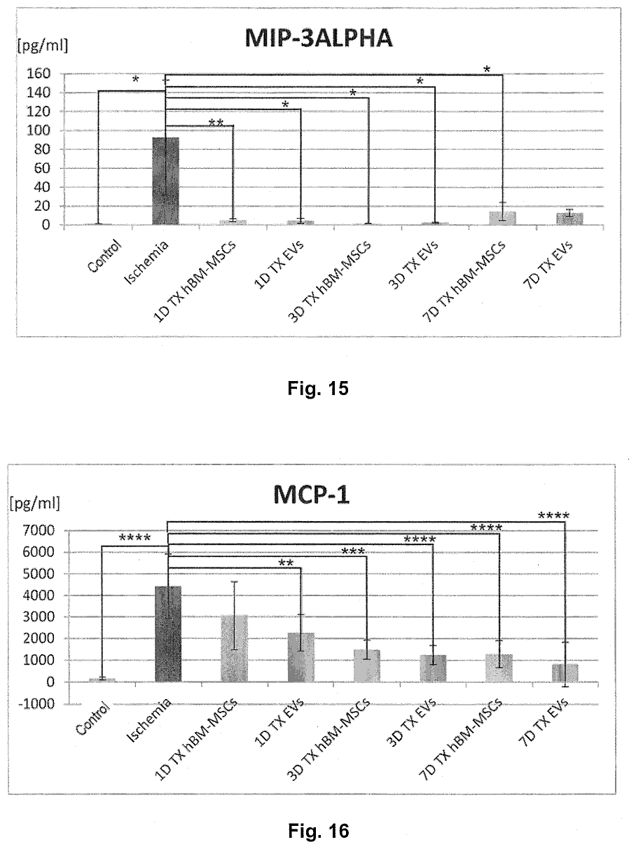

FIG. 15 shows the levels of macrophage inflammatory protein 3 alpha in the right brain hemisphere in individual experimental rat groups. Results are shown as mean values±SD, n=6, *<0.05; **p<0.01.

FIG. 16 shows the levels of monocyte chemotactic protein in the right brain hemisphere in individual experimental rat groups. Results are shown as mean values±SD, n=6, **p<0.01; ***p<0.001; ****p<0.0001.

In order to better illustrate the present invention, the microvesicles derived from mesenchymal stem cells for use as an immunomodulating medicament administered intra-arterially in treatment of neuroinflammation, in particular induced by stroke and the pharmaceutical composition for treatment of neuroinflammation, in particular induced by stroke, are demonstrated in the following examples. The scope of the invention however is not to be limited merely to the subject matter of the examples hereinbelow.

### EXAMPLE 1

### Preparation of Extracellular Vesicles (EVs) Released from Human Bone Marrow Derived Mesenchymal Stem Cells (hBM-MSCs)

Human mesenchymal stem cells isolated from bone marrow (hBM-MSCs) were used, from healthy adult donors of both sexes, commercially available (Lonza). Frozen hBM-MSCs provided in the number of 75×10⁴ cells constituting a second passage were cultured in vitro for amplification thereof until specific amounts were obtained as required for experimental procedures.

hMB-MSCs Cell Culture

Human bone marrow-derived mesenchymal stem cells (hBM-MSCs) (Lonza) were seeded in flasks having 75 cm² surface area with density of 3×10⁵ cells/flask and were cultured in MSCBM™ medium (Lonza) supplemented with 10% MCGS, L-glutamine and gentamicin in the atmosphere of air with 5% CO₂ content, 95% humidity and in temperature of 37° C. Culture medium was replaced with fresh medium every 2-3 days. In order to maintain a constant proliferation level, hBM-MSCs were passaged in 5-day intervals after reaching 70-80% confluent growth. Cells obtained from passages 4-6 were used for experiments.

Isolation of Extracelluler Vesicles (EVs) Released from hBM-MSCs During In Vitro Cell Culture

Extracellular vesicles released by hBM-MSCs during in vitro cell culture were isolated from their culture supernatants. For this purpose, in the conducted hBM-MSCs cultures showing 50% confluence, the MSCBM™ medium (Lonza) was replaced with fresh medium and cells were cultured further for 2-3 days in the atmosphere of air with 5% CO₂ content, 95% humidity and in temperature of 37° C. EVs were isolated from media obtained from culture with 5×10⁵ hBM-MSCs showing 90% confluent growth. For this purpose, supernatant from such cultures was collected and centrifuged twice, first at the speed of 200×g, for 10 minutes, and then at the speed of 500×g, for 10 minutes, in temperature of 4° C. to remove dead cells, and then being frozen in temperature of −70° C. Before conducting experiments, the supernatant with the contained EVs was thawed, centrifuged at the speed of 2000×g, for 20 minutes in temperature of 4° C. to remove dead cells and apoptotic vesicles, and then it was twice subjected to ultracentrifugation at the speed of 100 000×g in temperature of 4° C. for 75 minutes to obtain microvesicles. Isolated EVs were suspended in 100 μl of deionized PBS (BPBS, Lonza) and were either used for experiments or frozen in temperature of −70° C. for further experimental procedures.

FIG. 1 shows the obtained EVs from hBM-MSCs that were used in example 2.

### EXAMPLE 2

### Immunomodulating Activity of Extracellular Vesicles (EVs) Released from Human Bone Marrow-Derived Mesenchymal Stem Cells (hBM-MSCs) in Rat Model of Brain Stroke

Brain Stroke Model Via Striatum Damage with Ouabain in Rat

A model of cytotoxic brain damage corresponding to stroke developed by the present inventors was used for the experiments, the model involving a stereotactic administration of a Na⁺/K⁺ pump inhibitor (ouabain) to rat striatum (Janowski 2004). During surgical procedure, adult males of Wistar-herd rats were subjected to general anesthesia by intraperitoneal administration of ketamine (53 mg/kg) and medetomidine (0.4 g/kg). Animals under anesthesia were placed in a stereotaxic apparatus (Stoelting), had an incision made of the scalp along the sagittal suture and had a 0.5 cm diameter trepan hole drilled in the skull convexity, above the right hemisphere. Using a 5 μl Hamilton syringe with an end needle 15 mm in length and 33 mm in diameter, with an infusion pump, 1 μl of a 50 nmol ouabain solution (OUA) was administered at the speed of 0.5 μl/min to the striatum of the rat's right hemisphere (coordinates: A 0.5, L 3.8, D 4.7 mm; the reference points in the horizontal plane was the bregma). After ouabain administration, the injection needle remained in the rat's brain for the next 5 minutes in order to prevent backflow of the administered solution. After the needle was removed, wound edges on the skin were joined using a surgical thread. After surgery, the animals were subcutaneously administered an antibiotic (Baytril, 0.4 g/kg of rat body mass) and an analgesic (Rycarfa, 5 mg/kg of rat body mass).

lmmunohistochemical Analysis

The immunohistochemical analysis of the transplanted hBM-MSCs and EVs was performed in rats' brains for individual time intervals. Additionally, populations of immunologically active cells were evaluated in the brains of experimental animals of the host cells. To this end, organ sections were fixed in 4% paraformaldehyde for 15 minutes in room temperature and then blocked and permeabilized by adding a mixture of 10% GS, 0.25% Triton and 0.1% BSA (60 min, room temperature). Next, primary antibodies were added, specific for human antigens: mouse anti-human: CD44 (1:100), STEM121 (1:100) and recognizing rat antigens: mouse anti-rat: ED1 (1:100), CD45RA (1:100), CD5 (1:80), CD15 (1:20) and rabbit anti-rat: GFAP (1:200), the preparations were incubated for 24 h in temperature of 4° C. After the primary antibodies were washed off, secondary antibodies were added conjugated with Alexa 488 fluorochrome: goat anti-mouse or goat anti-rabbit of the corresponding isotype: IgG1, IgM or IgG(H+L) (1:500). After the secondary antibodies were washed off, cell nuclei were stained using 5 μM Hoechst 33258 (Life Technologies). In addition, Sudan Black was added to the rat brain sections in order to quench tissue autofluorescence. After the final wash, the preparations were preserved using Fluorescence Mounting Medium (Dako). The results obtained in the experiments were analysed using a confocal microscope (Zeiss LSM 780).

FIG. 2 shows the obtained results.

The analysis of selected immunologically active cells in rat brain demonstrated that due to striatum damage with ouabain there is an increase in numbers for some populations in comparison with healthy rats, including: GFAP⁺ astrocytes (42.1±7.2% vs 34.2±8.3%), microglial ED1⁺ cells (37.1±9.9% vs 11.1±6.8), CD45RA⁺ leukocytes (59.1±9.9% vs 44.4±10.4) and CD5 T lymphocytes (18.6±8.8% vs 7.1±4.1). In the case of neutrophils, no significant differences were observed between the number of CD15⁺ cells in rat brain after ouabain damage and in healthy rats, 24.8±3% vs 29.8±4.3% respectively.

Transplantation of hBM-MSCs or EVs in rats with striatum damage caused a reduction in astrocyte activation, in relation to animals after ouabain administration but not subjected to microvesicle transplantation or infusion. The reduction of the number of GFAP⁺ cells was evident 1, 3 and 7 days after cell or vesicle infusion, although only on the case of hBM-MSCs administration it was significant after 24 hours (FIG. 3). hBM-MSCs or EVs transplantation caused a decrease in microglial cells. The reduction in the number of ED1⁺ cells was highly statistically significant after 1, 3 and 7 days from transplantation. It appears interesting that this effect was more pronounced after administration of EVs as compared to hBM-MSCs (FIG. 4). Leukocyte analysis showed that an infusion of cells, as well as vesicles derived therefrom, leads to a decrease in the number of CD45RA⁺ cells in rat brain, while significance of the differences observed within the time interval varied slightly (FIG. 5). The number of T lymphocytes in hBM-MSCs or EVs transplant recipients was not changed. There were no statistically significant differences in animals administered with cells or vesicles, with the exception of rats evaluated on day 3 after transplantation of EVs, wherein an increase in the number of CD5⁺ was reported (FIG. 6). In all time intervals after hBM-MSCs or EVs transplantation there was a slight decrease in neutrophil numbers, but these differences in comparison to the animals without transplantation were not statistically significant (FIG. 7).

Analysis of Cytokine and Chemokine Levels in Rat Brain Homogenates Using the Luminex Apparatus

The analysis of the selected pro- and anti-inflammatory cytokine and chemokine levels was performed in homogenates obtained from rat brains using BioPlexPro kits specific for rat proteins utilizing the Luminex technology (BioRad). In order to perform the analysis, the studied samples of rat brain supernatants were diluted in ratio of 1:1 in buffer (Diluent Buffer) supplemented with 0.5% BSA, and lyophilisates of the standards were diluted in 500 μl of the same buffer and incubated for 30 minutes on ice. Magnetic beads were placed in 96-well plates, the beads coated with cytokines: IL-1α, IL-1β, IL-4, IL-6, IL-10, IFN-γ, TNFα and chemokines: CXCL1, MIP-1α, MIP-3α, MCP-1. After rinsing the plate twice with buffer (Rinse Buffer) as recommended by the manufacturer, the appropriately prepared standards or supernatants from rat brain homogenates were applied, each in triples. Such prepared plate was incubated in room temperature for 60 min, with constant mixing at the speed of 850 rpm/min. After this time, the plate contents were rinsed three times, a specific detection antibody was added and the plate was incubated in room temperature for the next 30 minutes, mixing at the speed of 850 rpm·min. Next, the plate was rinsed three times, a streptavidin-conjugated antibody was added and it was incubated in room temperature for 15 minutes, while mixing the plate contents at the speed of 850 rpm/min. After the plate was rinsed, buffer (Assay Buffer) was added and the reading was done using Luminex apparatus (BioRad).

Statistical Analysis

The obtained numerical results were presented as mean values of the individual experiments ±SD. In order to evaluate statistical significance for the differences between the mean values, a one-way ANOVA variance analysis was performed and the Bonferroni test. Prism 3.0 software was used for all calculations. Significance level below 0.05 was takes as statistically significant.

TABLE 1
Primary antibodies used in immunocytochemical
and immunohistochemical studies

| | | Manufacturer, |
|---|---|---|
| Antibody | Isotype, dilution | Catalog no. |
| mouse monoclonal | IgM, 1:20 | Serotec, MCA967 |
| anti-CD15 | | |
| mouse monoclonal | IgG1, 100 | Santa Cruz, sc-7297 |
| anti-CD44 | | |
| mouse monoclonal | IgG1, 1:100 | Serotec, MCA340R |
| anti-CD45RA | | |
| mouse monoclonal | IgG1, 1:80 | Serotec, MCA52R |
| anti-CD5 | | |
| mouse monoclonal | IgG1, 1:100 | BD Pharmingen, 556019 |
| anti-CD63 | | |
| mouse monoclonal | IgG3, 1:100 | Santa Cruz, sc-32299 |
| anti-CD73 | | |
| mouse monoclonal | IgG2b, 1:100 | Santa Cruz, sc-166028 |
| anti-CD81 | | |
| mouse monoclonal | IgG1, 1:100 | BD Pharmingen, 555370 |
| anti-CD9 | | |
| mouse monoclonal | IgG1, 1:100 | Santa Cruz, sc-59398 |
| anti-CD90 | | |
| mouse monoclonal | IgG1, 1:100 | Serotec, MCA341R |
| anti-ED1 | | |
| rabbit polyclonal | IgG(H + L), 1:200 | Dako, Z 0334 |
| anti-GFAP | | |
| mouse monoclonal | IgG1, 1:100 | Cellartis, Y40410 |
| anti-STEM121 | | |

TABLE 2
Secondary antibodies used in immunocytochemical
and immunohistochemical studies

| | | | Manufacturer, |
|---|---|---|---|
| Antibody | Fluorophore | Isotype, dilution | Catalog no. |
| goat anti-mouse | Alexa 488 | IgG1, 1:500/1:1000 | Invitrogen, |
| Alexa Fluor | | | A21121 |
| goat anti-mouse | Alexa 488 | IgG2b, 1:500/1:1000 | Invitrogen, |
| Alexa Fluor | | | A21141 |
| goat anti-mouse | Alexa 488 | IgG3, 1:500/1:1000 | Invitrogen, |
| Alexa Fluor | | | A21151 |
| goat anti-mouse | Alexa 488 | IgM, 1:500/1:1000 | Invitrogen, |
| Alexa Fluor | | | A21042 |
| goat anti-rabbit | Alexa 488 | IgG(H + L), | Invitrogen, |
| Alexa Fluor | | 1:500/1:1000 | A11008 |

The analysis of cytokine levels in rat brain homogenates using the BioPlexPro kit with Luminex technology (BioRad) showed that as a result of brain damage with ouabain there occurs a statistically significant activation of pro-inflammatory cytokines: IL-1α (α<0.01), IL-1β (α<0.0001), IL-6 (α<0.0001), an increase in IL-4 and IFN-γ levels and a decrease for IL-10. The changes resulting from ischemia led to a statistically significant increase in chemokine levels: CXCL1 (α<0.0001), MIP-1alpha (α<0.0001), MIP-3alpha (α<0.05) and MCP-1 (α<0.0001).

The results of studies performed in rats after striatum damage with ouabain showed that a transplant of hBM-MSCs or EVs leads to a reduction in pro-inflammatory cytokine levels. As a result of the transplantation, there is a reduction in activated interleukin 1α, this being the most pronounced after day 1 from transplantation (FIG. 8). The administration of cells and extracellular vesicles causes a decrease of the amounts of secreted: IL-1β and IL6. This reduction in cytokine levels is highly significant at all time points (FIG. 9, FIG. 10). In hBM-MSCs or EVs transplant recipients, there is also a reduction in IL-4 observed. Interestingly, on day 1 and 7 after EVs administration, the IL-4 level is much lower than after cell transplantation (FIG. 11). A similar effect of hBM-MSCs or EVs transplantation was reported in the case of IFN-γ, although a reduction in the amount thereof was not that spectacular (FIG. 12).

The analysis of chemokine levels in the brains of experimental animals showed a reduction in all of the studied factors after hBM-MSCs or EVs transplantation. The differences were highly statistically significant at all time points (FIGS. 13-16). In the case of MIP-3alpha, the administration of cells or vesicles isolated therefrom caused a reduction in chemokine levels to values observed in healthy rat brains. It should be emphasized that the effect of the transplanted EVs activity on the reduction of MIP-1α and MCP-1 amounts in relation to the values in animal brains after ischemia is much stronger than in comparison to hBM-MSCs transplantation.

1. Microvesicles derived from mesenchymal stem cells for use as an immunomodulating medicament administered intra-arterially in treatment of neuroinflammation, in particular induced by stroke.

2. Microvesicles for use according to claim 1, characterized in that the medicament is administered intra-arterially while performing a procedure of endovascular clot removal or during a later period or in patient who does not have the procedure of clot removal performed.

3. Microvesicles for use according to claim 2, characterized in that they are used for inducing one of the following immunomodulating effects: reduction of the number of inflammatory cells such as: activated microglia (ED1) and leucocytes (CD45) or proinflammatory molecules such as: IL-1alpha, IL-1beta, IL-6, IL-4, IFN-Gamma, CXCL1, MIP-1alpha, MIP-3alpha and MCP-1.

4. Microvesicles for use according claim 1, characterized in that they are extracellular vesicles (EVs) released from human mesenchymal stem cells, in particular from bone marrow (hBM-MSCs).

5. A pharmaceutical composition for treatment of encephalitis, in particular induced by stroke, characterized in that it comprises microvesicles derived from stem cells, wherein the composition is for intra-arterial administration.

6. The pharmaceutical composition according to claim 5, characterized in that it comprises microvesicles for use as defined in any of claims 1 to 4.
